(19) United States
(12) Patent Application Publication
(10) Pub. No.: US 2014/0235558 A1
(43) Pub. Date: Aug. 21, 2014

(54) PHARMACEUTICAL COMPOSITION HAVING ACTIVITY OF ANTICANCER

(71) Applicant: Hanall Biopharma Co., Ltd., Daejeon (KR)

(72) Inventors: Sung-Wuk Kim, Seongnam-si (KR); Sung-Soo Jun, Seongnam-si (KR); Chang-Hee Min, Seoul (KR); Ja-Seong Koo, Daejeon (KR); Jin-Wook Kim, Daejeon (KR); Yong-Eun Kim, Daejeon (KR); Sang-Ouk Sun, Daejeon (KR)

(21) Appl. No.: 13/930,800

(22) Filed: Jun. 28, 2013

Related U.S. Application Data
(63) Continuation-in-part of application No. 14/004,746, filed on May 7, 2014.

(30) Foreign Application Priority Data
Jun. 15, 2010 (KR) 10-2010-0056726
Aug. 13, 2010 (KR) 10-2010-0078338
Jun. 14, 2011 (KR) 10-2011-0057664

(51) Int. Cl. A61K 31/155; A61K 45/06; A61K 31/506; A61K 31/7004

(52) U.S. Cl. CPC A61K 31/155; A61K 31/7004; A61K 45/06; A61K 31/506
USPC 514/23; 514/635

(57) ABSTRACT

Disclosed is an anticancer pharmaceutical composition comprising phenformin or a pharmaceutically acceptable salt thereof, and a glycolysis inhibitor, particularly, 2-deoxy-D-glucose as active ingredients. These ingredients act in synergy with each other, thus exhibiting more potent inhibitory activity against the growth of cancer cells, compared to individual ingredients. Also, the synergistic anticancer activity allows the individual drugs to be used in lower amounts, which leads to a reduction in the occurrence of adverse effects. In addition, the time-lag release or administration of the ingredients decreases blood lactic acid levels to significantly mitigate the adverse effect of lactic acidosis, as well as exerting high anticancer effects. Particularly, the pharmaceutical composition can be formulated to dosage forms effective for therapy, increasing the drug compliance of the subject.

### TECHNICAL FIELD

The present invention relates to a pharmaceutical composition with anticancer activity. More particularly, the present invention relates to a novel anticancer composition, comprising phenformin or a pharmaceutically acceptable salt thereof, and a glycolysis inhibitor, particularly, 2-deoxy-D-glucose, which exerts excellent therapeutic activity for cancer with a significant reduction in side effects. Also, the present invention is concerned with a method for treating cancer, comprising a therapeutically effective amount of the composition to a subject in need thereof.

### BACKGROUND ART

Phenformin was discovered as an oral antidiabetic drug in the late 1950s. In expectation of its ability to effectively lower blood glucose levels and prevent the onset of complications of diabetes without provoking hypoglycemia or hyperinsulinemia, phenformin, which is a kind of biguanide drug like metformin, was applied to the therapy of insulin-non-dependent diabetes (type II diabetes), but its usage was completely banned in the late 1970s because of the severe side effect of causing lactic acidosis.

However, phenformin has been studied and evidenced for anticancer activity as biguanide drugs are known to be effective for the therapy of p53 gene-deficient cancer thanks to its ability to activate AMPK (AMP-activated protein kinase), an enzyme playing a crucial role in the physiological regulation of carbohydrate and lipid metabolism (Effect of phenformin on the proliferation of human tumor cell lines. Life sciences, 2003 Dec. 19:vol74(issue 5):643-650.) (Potentiation of antitumor effect of cyclophosphamide and hydrazine sulfate by treatment with the antidiabetic agent, 1-phenylethylbiguanide (phenformin), Cancer let. 1979 October; 7(6):357-61).

So far, however, phenformin has not been developed as an anticancer agent because its potential to cause lactic acidosis, the greatest problem with phenformin, still remains unsolved.

Glycolysis inhibitors are known for their anticancer activity by inhibiting enzymes involved in the glycolysis pathway of cancer cells. The glycolysis pathway in cancer cells is largely divided into glucose transport, capture by phosphorylation, conversion into biosynthetic intermediates, and release steps. A glycolysis inhibitor acts to inhibit the production of enzymes involved in the glycolysis pathway, including GLUT1 (glucose transporter 1), GLUT2 (glucose transporter 2), HK2 (hexokinase-2), PFK1 (phosphofructokinase type 1), PKM2 (pyruvate kinase), LDHA (lactate dehydrogenase A), MCT1 (monocarboxylate transporter 1), or MCT4 (monocarboxylate transporter 4).

Particularly, 2-deoxy-D-glucose, a glucose derivative and a representative glycolysis inhibitor, functions to restrain cancer cells from sugar uptake, thus inhibiting the growth of cancer cells. Tumor cells require energy for supporting their rapid proliferation and expansion. In addition, even the cancer cells that more slowly proliferate in the hypoxic area of tumor require energy. Increased cellular uptake of glucose is one of the most common features of highly malignant tumors. Therefore, the inhibition of anaerobic glycolysis by 2-deoxy-D-glucose is useful as a means for killing cancer cells. In addition, 2-deoxy-D-glucose has recently been found to activate AMPK, like biguanide drugs.

In spite of such pharmaceutical effects, the drugs are prone to provoking adverse effects since it is difficult to sufficiently reduce the content or dose of each drug, or when used individually, they exhibited only limited therapeutic effects. These limitations serve as barriers to suggest or develop drugs therapeutically effective for the treatment of cancer-related diseases. Particularly, there have been neither examples of the simultaneous use of phenformin and 2-deoxy-D-glucose in the treatment of cancer, nor effects thereof, so far.

### DISCLOSURE

### Technical Problem

Leading to the present invention, intensive and thorough research into an anticancer pharmaceutical formulation, resulted in the finding that phenformin or a pharmaceutically acceptable salt thereof acts in synergy with a glycolysis inhibitor, e.g., 2-deoxy-D-glucose, exerting a surprisingly increased anticancer effect, and that when used in combination, their doses necessary for the therapy of cancer can be decreased, thus reducing adverse effects.

The present invention addresses a pharmaceutical composition and a method for the treatment of various cancers.

Also, the present invention addresses a method for treating cancer, comprising administering to a subject an effective amount of a combination of phenformin or a pharmaceutically acceptable salt thereof, and a glycolysis inhibitor.

In one embodiment, the drugs may be administered simultaneously or with a time lag. Hence, the present invention envisages a method for treating cancer, using a simple combination agent configured to administer phenformin or a pharmaceutically acceptable salt thereof, and a glycolysis inhibitor, preferably 2-deoxy-D-glucose, or a timed-release agent configured to release the drugs with a time lag, thereby exerting improved anticancer activity with a reduction in adverse effects.

Also, the present invention provides a method for treating cancer, comprising administering an anticancer agent in addition to phenformin and a glycolysis inhibitor by which a therapeutically synergistic effect can be obtained in various aspects.

It is an object of the present invention to provide a pharmaceutical composition for the therapy of cancer, comprising phenformin or a pharmaceutically acceptable salt thereof, and a glycolysis inhibitor as active ingredients. In the pharmaceutical composition for the therapy of cancer, phenformin and a glycolysis inhibitor may be used as sole active ingredients or together with another active ingredient.

In one embodiment, the present invention addresses a pharmaceutical composition comprising an anticancer agent in addition to a combination of phenformin or a pharmaceutically acceptable salt thereof, and a glycolysis inhibitor.

It is another object of the present invention to provide a method for treating cancer, comprising administering phenformin or a pharmaceutically acceptable salt thereof, and a glycolysis inhibitor in a therapeutically effective amount to a subject in need thereof.

According to one embodiment, a therapeutically effective amount of glycolysis inhibitor is administered to a subject, followed by a therapeutically effective amount of phenformin or a pharmaceutically acceptable salt thereof.

In another embodiment, a pharmaceutical composition comprising an immediate release form of a glycolysis inhibitor and a time release form of phenformin or a pharmaceutically acceptable salt thereof is administered in a therapeutically effective amount to a subject.

Preferably, the glycolysis inhibitor is 2-deoxy-D-glucose.

### Technical Solution

A detailed description will be given of the pharmaceutical composition and method for the treatment of cancer in accordance with the present invention, below.

Unless stated otherwise, the term “pharmaceutical composition,” as used herein, is intended to encompass a single dose form and a multiple-dose form, whether oral or non-oral, which are configured to be administered at once, and in a divided manner of two or more rounds respectively. For example, “a pharmaceutical composition comprising phenformin hydrochloride and a glycolysis inhibitor” may be in the form of a single dose unit containing the two or more active ingredients together, or in the form of two or more dose units containing the two or more active ingredient respectively. In addition, even a single dose unit form containing the two active ingredients together may be configured to release the active ingredients with a time lag in the body. When the pharmaceutical composition is in the form of two dose units corresponding to the two active ingredients, they may be administered with a time lag therebetween. Alternatively, the two dose units may be administered simultaneously if they are configured to release the two active ingredients with a time lag therebetween. Like this, when the two active ingredients exert a synergistic effect together, any pharmaceutical composition, whether in the form of a single dose unit or two dose units, falls within the “pharmaceutical composition comprising phenformin hydrochloride and a glycolysis inhibitor.”

The two active ingredients may be released or administered simultaneously or with a time lag therebetween.

Hence, the pharmaceutical composition for the treatment of cancer, comprising phenformin or a pharmaceutically acceptable salt thereof, and a glycolysis inhibitor as active ingredients may be a simple combination pharmaceutical composition in which the two ingredients are co-administered (or co-released) simultaneously, or a pharmaceutical composition in which the two ingredients are co-administered (or co-released) with a time lag therebetween.

Preferably, the pharmaceutical composition is configured to administer or release the active ingredients with a time lag therebetween, exhibiting more enhanced anticancer activity with a significant reduction in lactic acidosis, a most problematic side effect of phenformin.

Also, contemplated in accordance with another embodiment of the present invention are a pharmaceutical composition comprising phenformin or a pharmaceutically acceptable salt thereof, and a glycolysis inhibitor, preferably 2-deoxy-D-glucose as active ingredients which is configured to release the active ingredients with a time lag therebetween, and a time-lag administration method of the active ingredients.

The “time-lag,” as used herein in the context of administration or release, is intended to encompass the release or administration of active ingredients so as to allow the active ingredients to be absorbed sequentially, but not simultaneously, into the body. The “time-lag release or time-lag administration” is applied to a combination formulation in which individual active ingredients are contained together within a single dose units, as well as a formulation in which individual active ingredients are in the form of different respective dose units if the formulation is configured to release the active ingredients in a time lag pattern even when they are administered simultaneously. Also, the time-lag administration” is true of the administration intended to provide the two active ingredients at regular time intervals.

In one embodiment thereof, the present invention provides a pharmaceutical composition for the treatment of cancer, comprising a representative glycolysis inhibitor, 2-deoxy-D-glucose, represented by the following Chemical Formula 1, and phenformin, represented by the following Chemical Formula 2, or a pharmaceutically acceptable salt thereof as active ingredients:

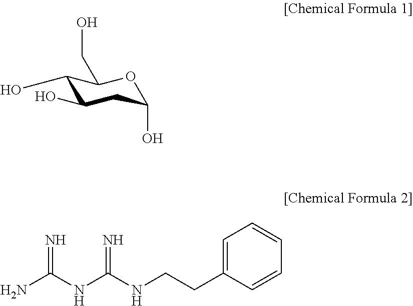

Concrete examples of the diseases to which the pharmaceutical composition of the present invention is applicable include uterine cancer, breast cancer, stomach cancer, brain cancer, rectal cancer, colon cancer, lung cancer, skin cancer, blood cancer, and liver cancer, with preference for breast cancer, stomach cancer or colon cancer.

Experiment data demonstrated that a combination of phenformin and 2-deoxy-D-glucose, a glycolysis inhibitor, had even higher inhibitory activity against the growth of tumor cells, compared to individuals or a combination of one of the active ingredients with a different ingredients, when used in the same amount. This high synergistic effect is believed to be attributed to the following events.

In the present invention, the glycolysis inhibitor may be an agent that serves to inhibit, retard, attenuate or diminish the glycolysis pathway of glucose metabolisms in cancer cells.

In addition, the glycolysis inhibitor may be an agent that serves to inhibit, retard, attenuate or diminish the activity of at least one of the enzymes involved in the glucose metabolism of cancer cells, including GLUT1 (glucose transporter 1), GLUT2 (glucose transporter 2), HK2 (hexokinase-2), PFK1 (phosphofructokinase type 1), PKM2 (pyruvate kinase), LDHA (lactate dehydrogenase A), MCT1(monocarboxylate transporter 1) and MCT4 (monocarboxylate transporter 4).

Examples of the glycolysis inhibitor useful in the present invention may include, but are not limited to, 2-deoxy-D-glucose (2-DG), 2-fluoro-deoxyglucose, 3-bromopyruvate (3-BrPA), 3-bromopyruvate propyl ester (3-BrOP), 5-thioglucose, iodoacetate, lonidamine, oxythiamine, and dichloroacetic acid (DCA).

Moreover, a tyrosine kinase inhibitor, such as imatinib, may be used as a glycolysis inhibitor in the present invention. For example, Bcr-Abl tyrosine kinase, such as imatinib; an RAS inhibitor; an RAF inhibitor, such as sorafenib, vemurafenib, or dabrafenib; an MEK or ERK inhibitor, such as trametinib or MEK-162, may be used as a glycolysis inhibitor.

Preferably, the glycolysis inhibitor may be 2-deoxy-D-glucose.

In the glycolysis pathway responsible for producing energy (ATP) necessary for the growth and homeostasis of cells from the D-glucose, 2-deoxy-D-glucose interferes with the isomerization of glycose 6-phosphate to fructose 6-phosphate, thus blocking the energy supply of cancer cells. In addition, 2-deoxy-D-glucose is known to activate AMPK (AMP-activated protein kinase) in combination with a biguanide drug, although weakly.

Phenformin functions to activate AMPK to inhibit the activity of mTOR (mammalian target of rapamycin), an enzyme regulating protein synthesis, which in turn, deactivates S6K1, thereby suppressing the growth of cancer cells. In addition, it can inhibit the growth of cancer cells through a different mechanism in which it inhibits the production of NAD+ in complex I of the mitochondrial oxidative phosphorylation pathway, which is responsible for the synthesis of the energy source ATP, thus restraining energy generation.

However, the blockage of the energy supply to cancer cells and the activation of AMPK by the sole administration of 2-deoxy-D-glucose are insufficient for the therapy of cancer. Cancer cells are known to have an energy supply route via glutamine in addition to the glycolysis pathway of glucose. Further, the amount of 2-deoxy-D-glucose necessary to reach the AMPK activation effective for anticancer activity is too large to consume. For these reasons, 2-deoxy-D-glucose alone has not been developed thus far. Phenformin, when administered alone, cannot effectively block the supply of energy to cancer cells, and in addition is insufficient to provoke desired AMPK activation. For this reason, phenformin has not been developed as an anticancer agent.

The present inventors have studied the co-administration of 2-deoxy-D-glucose and phenformin to develop an anticancer composition, and surprisingly found that much higher anticancer activity was obtained when 2-deoxy-D-glucose and phenformin were co-administered than when either of them was used solely. Accordingly, the pharmaceutical composition of the present invention is expected to exert a therapeutically synergistic effect on various cancers.

Besides, phenformin is known to activate glycolysis in a hypoxic or anaerobic condition to increase blood lactic acid levels, causing lactic acidosis. The problem of lactic acidosis makes it difficult to use phenformin as an anticancer drug.

With the side effect of lactic acidosis in mind, the present inventors continued to research the use of phenformin, and the research culminated in finding that when phenformin was administered or released with a time lag after 2-deoxy-D-glucose was absorbed, only a significantly reduced level of lactic acid was detected, without the generation of lactic acidosis because absorption of 2-deoxy-D-glucose suppressed anaerobic glycolysis in advance.

Hence, the pharmaceutical composition comprising phenformin or a pharmaceutically acceptable salt thereof, and 2-deoxy-D-glucose as active ingredients according to one embodiment of the present invention is preferably configured to allow the active ingredients to be released or administered, with the aim of obtaining a synergistic anticancer effect, and a significant reduction in the main problem with phenoformin, lactic acidosis.

The time lag may be preferably 0.25 to 4.0 his, and more preferably 0.5 to 2.0 hrs. In a preferred embodiment, of the two active ingredients, 2-deoxy-D-glucose may be released or administered in advance.

When they are released or administered with a time lag exceeding the range, phenformin or 2-deoxy-D-glucose may be reduced in bioavailability, and a synergistic effect attributable to the time lag cannot be obtained. When phenformin or a pharmaceutically acceptable salt thereof is released or administered in advance of 2-deoxy-D-glucose, it is impossible to allow the absorption of phenformin or a pharmaceutically acceptable salt thereof after the sufficient suppression of anaerobic glycolysis and thus to effectively reduce the side effects.

When the pharmaceutical composition according to one embodiment of the present invention is administered, the active ingredients act in synergy with each other, so that each of the active ingredients can be used in a significantly decreased amount, which leads to a reduction in side effects while exerting higher therapeutically synergistic effects.

In the pharmaceutical composition, phenformin may be used as it is, or may be in the form of an inorganic acid addition salt such as hydrochloride, or an organic acid addition salt such as besylate and acetate, in consideration of solubility and stability. More preferred is phenformin hydrochloride.

A single dose of the pharmaceutical composition may comprise phenformin hydrochloride in an amount of from 10 to 1,000 mg, preferably in an amount of from 20 to 200 mg, and more preferably in an amount of from 25 to 150 mg, and 2-deoxy-D-glucose in an amount of from 10 to 4,000 mg, preferably in an amount of from 100 to 2,500 mg, and in an amount of from 100 to 1,000 mg, and may be administered once or multiple times per day. In an alternative embodiment, the pharmaceutical composition may comprise phenformin hydrochloride and 2-deoxy-D-glucose preferably at a weight ratio of from 1:400 to 100:1, and more preferably at a weight ratio of from 1:200 to 10:1.

When the weight ratio exceeds the lower or upper limit, the effect obtained from each of the active ingredients may not reach a desired level, or a side effect may be evoked by an excess of one of the active ingredients. In addition, at a weight ratio exceeding either of the limits, it may be difficult to administer the composition because its own weight is too large. That is, such a composition is too poor in drug compliance to effectively serve as a pharmaceutical composition.

In another embodiment, the pharmaceutical composition of the present invention may comprise at least one pharmaceutically acceptable carrier in addition to the active ingredients.

As used herein, the term “pharmaceutically acceptable carrier” means a pharmaceutical additive that is useful in formulating the pharmaceutical composition into dosage forms and does neither produce toxicity nor irritation in the condition of practical use. Concrete contents of this additive may be determined depending on various factors including solubilities and chemical properties of the active ingredients used, and administration routes, or according to standard pharmaceutical modalities.

In greater detail, the pharmaceutical composition of the present invention may be formulated, together with a pharmaceutical additive, such as an diluent as a pharmaceutically acceptable carrier, a disintegrant, a sweetener, a binder, a coating agent, a swelling agent, a lubricant, an aromatic, etc. into forms suitable for desired administration routes. The amount of the carrier needed per administration unit may be sufficiently large to provide the dose size and form which guarantees the drug compliance of the subject.

The formulation of the pharmaceutical composition may be in an oral or non-oral form, as typified by, but not limited to, tablets (press-coated tablets, coated tablets, multiple layer tablets, etc.), fine particles, capsules containing liquid or powders, pills, granules, powders, troches (inclusive of liquid-filled), chews, multi- and nano-particles, gels, solid solutions, liposomes, films (inclusive of mucous adhesive), ovules, sprays, and liquid. Examples of the liquid include suspensions, solutions, syrups, and elixirs, but not limited thereto.

Of the oral forms, a typical tablet may comprise a disintegrant in addition to the active ingredients. Examples of the disintegrant include, but are not limited to, starch or modified starch, such as sodium starch glycolate, corn starch, potato starch or pregelatinized starch; clay, such as bentonite, montmorillonite, or beegum; celluloses, such as low-substituted hydroxypropyl cellulose; alginates, such as sodium alginate or alginic acid; cross-linked cellulose such as croscarmellose sodium; cross-linked polymers such as crospovidone; effervescent agents such as sodium bicarbonate, citric acid, etc. and a combination thereof.

The disintegrant may be preferably used in an amount of from about 0.5 wt % to about 30 wt %, based on the total weight of the dosage form, and more preferably in an amount of from about 1 wt % to about 20 wt %.

In addition, a tablet may further comprise a binder to provide adhesiveness. Illustrative, but non-limitative examples of a binder useful in the present invention include gelatin, sugar, natural or synthetic gums, polyvinylpyrrolidone (povidone), polyvinylalcohol, copovidone, starches, hydroxypropyl cellulose, and hypromellose.

The binder may be used preferably in an amount of from about 0.1 wt % to about 40 wt %, based on the total weight of the dosage form, and more preferably in an amount of from about 0.5 wt % to about 25 wt %.

In addition, a tablet may further contain a diluent. As the diluent, starch, microcrystalline cellulose, lactose, glucose, mannitol, alginate, alkaline earth metal, polyethylene glycol, or dicalcium phosphate may be used.

The amount of the diluent may be preferably on the order of from about 0.5 wt % to about 90 wt %, based on the total weight of the dosage form, and more preferably on the order of 2 wt % to 75 wt %.

Another additive that may be contained in the tablet is a lubricant. Examples of the lubricant include talc, stearic acid, magnesium stearate, calcium stearate, zinc stearate, sodium stearyl fumarate, hydrogenated vegetable oil, and polyethylene glycol, but are not limited thereto.

Based on the total weight of the dosage form, the lubricant may be used preferably in an amount of from about 0.1 wt % to about 20 wt %, and more preferably in an amount of from about 0.2 wt % to about 10 wt %.

Optionally, the tablet may comprise a surfactant such as sodium lauryl sulfate or polysorbate 80, and a glidant such as colloidal silicon dioxide, silica hydrated silica or talc.

Preferably, each of the surfactant and the glidant may range in content from about 0.1 wt % to about 20 wt %, based on the total weight of the dosage form.

Other additives in the formulation according to the present invention may be typified by an antioxidant, a colorant, a flavorant, a preservative, and a taste-masking agent.

As described above, a tablet may be formed by compressing all of the employed ingredients directly or through a roller. Alternatively, the ingredients contained in the tablet may be wet-, dry- or melt-granulated, or melt-congealed, or compressed prior to a tableting process. The final formulation form may include at least one layer, or may or may not be coated, or may be capsulated.

In addition, the composition of the present invention may be formulated to various release forms which can be classified into immediate and modified release forms according to the time of release. Among the modified release forms are delayed-, sustained-, pulsed-, controlled-, targeted- and programmed release forms.

In accordance with another aspect thereof, the present invention addresses a pharmaceutical composition for the therapy of cancer, comprising 2-deoxy-D-glucose, and phenformin or a pharmaceutically acceptable salt thereof as active ingredients, wherein 2-deoxy-D-glucose is released in advance of phenformin or a pharmaceutically acceptable salt thereof.

Featuring the release or administration of the active ingredients with a time lag therebetween, as described above, the pharmaceutical composition of the present invention enjoys the advantage of significantly increasing the anticancer activity of each of the ingredients, and significantly reducing lactic acidosis, a main problem with phenformin.

To this end, a pharmaceutical composition comprising 2-deoxy-D-glucose may be preferably formulated to an immediate (fast) release form while a pharmaceutical composition comprising phenformin may be in a delayed release form, e.g. sustained- or pulsed-release form. Alternatively, a pharmaceutical composition may be formulated to a unit dosage form which is configured to release the active ingredients with a time lag therebetween. In this regard, the time lag-release form may be preferably designed to release 2-deoxy-D-glucose, followed by the absorption of phenformin into the body.

Like this, the reason why 2-deoxy-D-glucose is allowed to be released and absorbed in advance of phenformin is attributed to the mechanism of action described above and may be elucidated as follows.

Rapid solubilization and absorption is advantageous for the bioavailability of such a drug as 2-deoxy-D-glucose. In contrast, a biguanide drug such as phenformin is apt to undergo a rapid change in blood level because of its high loss rate, thereby provoking an adverse effect and resistance thereto. In practice, adverse effects associated with biguanide drugs occasionally occur in the gastrointestinal tract, as exemplified by anorexia, vomiting, and diarrhea. In addition to the adverse effects, the rapid release of phenformin may cause an excessive decrease in blood sugar level.

For a patient's convenience and to enhance therapeutic effects, a pharmaceutical composition comprising 2-deoxy-D-glucose is preferably formulated to an immediate release form while a pharmaceutical composition comprising phenformin is in a delayed release form, e.g. sustained or pulsed release form.

So long as it is sufficient to allow 2-deoxy-D-glucose to act not simultaneously with, but prior to phenformin or a pharmaceutically acceptable salt thereof, any time lag of release between the two active ingredients may be applied to the present invention. Preferably, phenformin or a pharmaceutically acceptable salt thereof is released at 0.25 to 4.0 hrs, and more preferably at 0.5 to 2.0 hrs after the commitment of release of 2-deoxy-D-glucose.

Since 2-deoxy-D-glucose is absorbed immediately after release, the time of 0.25 hrs after the commitment of release of 2-deoxy-D-glucose is sufficiently long to elicit a time lag effect for phenformin. When the time lag is over 4 hrs, however, a formulation containing phenformin or a pharmaceutically acceptable salt thereof is highly likely to proceed to the small intestine, suffering from the disadvantage of decreasing in bioavailability and being difficult to pharmacokinetically embody.

2-Deoxy-D-glucose is water soluble and can rapidly be eluted. Thus, the formulation is preferably configured to release 2-deoxy-D-glucose in an amount of 80.0% or more based on the total weight of 2-deoxy-D-glucose within 0.05 to 1 hr after the commitment of release.

In an alternative preferred embodiment, the formulation is designed to release the phenformin or a pharmaceutically acceptable salt thereof in an amount of 80.0 wt % or more of its total weight within 0.25 to 12.0 hrs after the commitment of the release thereof with such a time lag as is described.

In detail, when phenformin or a pharmaceutically acceptable salt thereof is in the form of a pulsed release formulation, it is preferred that phenformin or a pharmaceutically acceptable salt thereof be released in an amount of 80.0% of the total weight thereof within 0.25 hrs after the commitment of release because a pulsed release formulation must perform release and elution almost simultaneously. For a sustained formulation, phenformin or a pharmaceutically acceptable salt thereof may be released in an amount of 80.0% or more of the total weight thereof within about 12 hrs after the commitment of release.

When phenformin or a pharmaceutically acceptable salt thereof takes a pulsed or sustained release formulation, no particular limitations are imparted to the release conditions which guarantee the formulation to exhibit the above-mentioned release properties.

In order to exhibit the time-lag release properties therethrough, an enteric-coated formulation is subjected to an elution test for 2 hrs in 0.1N HCl (simulated gastric fluid) and then further in a phosphate buffer, pH 6.8 (simulated intestinal fluid). If it is immediately eluted at pH 6.8, the time lag cannot be identified. This condition is made in consideration of the passage order and gastric retention time of a drug after the oral administration of the drug to a human. Since a time lag in vitro is simulated in vivo, a release property which is determined to be suitable in vitro is applicable to an in vivo condition.

For use in formulating a sustained or pulsed release tablet, a matrix base is not specifically limited, but may be selected from the group consisting of an enteric coating polymer, a hydrophobic material, a hydrophilic polymer, and a combination thereof.

Examples of the enteric coating polymer include, but are not limited to, polyvinylacetate phthalate, polymethacrylate copolymers, such as poly(methacrylate, methylmethactylate) copolymer, and poly(methacrylic acid, ethylacrylate) copolymer, hypromellose phthalate, hypromellose acetate succinate, shellac, cellulose acetate phthalate, and cellulose propionate phthalate.

The hydrophobic material must be pharmaceutically acceptable, and may be exemplified by, but not limited to, polyvinyl acetate, a polymethacrylate copolymer, such as poly(ethylacrylate, methyl methacrylate) copolymer, and poly(ethylacrylate, methyl methacrylate, trimethylammonioethylmetachrylate)copolymer, ethyl cellulose, cellulose acetate, fatty acids, fatty acid esters, fatty acid alcohols, waxes, and inorganic materials.

In greater detail, the fatty acids or the fatty acid esters are selected from among glyceryl palmitostearate, glyceryl stearate, glyceryl behenate, cetyl palmitate, glyceryl monooleate, and stearic acid. Within the scope of the fatty acid alcohols, cetostearyl alcohol, cetyl alcohol, and stearyl alcohol may fall. As the waxes, carnauba wax, beeswax, and microcrystalline wax. The inorganic materials may include talc, precipitated calcium carbonate, calcium monohydrogen phosphate, zinc oxide, titanium oxide, kaolin, bentonite, montmorillonite, and beegum.

Turning now to the hydrophilic polymers, their examples include sugars, cellulose derivatives, gums, proteins, polyvinyl derivatives, polyethylene derivatives, and carboxyvinyl polymers, but are not limited thereto.

Dextrin, polydextrin, dextran, pectin and pectin derivatives, alginate, polygalacturonic acid, xylan, arabinoxylan, arabinogalactan, starch, hydroxypropyl starch, amylose, and amylopectin are examples of the sugars useful in the present invention. As the cellulose derivatives, hypromellose, hydroxypropyl cellulose, hydroxymethyl cellulose, hydroxyethyl cellulose, methyl cellulose, carboxy methylcellulose sodium, and hydroxyethylmethylcellulose may be used. As for gums, their examples are guar gum, locust bean gum, tragacanth, carrageenan, gum acacia, gum arabic, gellan gum and xanthan gum. The protein may be selected from among gelatin, casein and zein. As polyvinyl derivatives, polyvinyl alcohol, and polyvinyl pyrrolidone are available. The polyethylene derivative may be typified by polyethylene glycol and polyethylene oxide. Carbomer is suitable as a carboxyvinyl polymer.

The formulation of the pharmaceutical composition and the range of the additive that can be used in the present invention are not limited to the above-mentioned those, and can be suitably selected by those skilled in the art.

Preferably, as will be explained in the following Examples 9 to 11, the pharmaceutical composition may be formulated to a Press-coated tablet comprising early release granules of 2-deoxy-D-glucose and a late release inner core of phenformin or a pharmaceutically acceptable salt thereof. In such a Press-coated tablet constitution, it is easy to release the ingredients with a time lag, and to control the elution rate of the core of phenformin.

Alternatively, a single formulation containing phenformin or a pharmaceutically acceptable salt thereof is coated to release the ingredient in a retarded pattern, so that a time lag release can be achieved even when it is administered simultaneously with a single formulation of 2-deoxy-D-glucose.

The effective dosage for the therapy of various cancers of the pharmaceutical composition may vary depending on various factors, including the kind of disease to be treated, the patient's age, weight, state of health, gender and diet, the time of administration, the route of administration, the blood clearance rate of the composition, the duration of administration, the drug to be used together, etc. In general, it may be administered in a single dose or in multiple doses per day at a daily dose ranging from 20 to 5,000 mg. It is obvious to those skilled in the art that the dose of each active ingredient must not be high sufficient to evoke an adverse effect.

Formulation to various oral dosage forms, and immediate release forms, sustained release forms, pulsed release forms, or time-lag release forms can be achieved using any known method that allows the active ingredients to be released with such a time lag as described above.

In another preferred embodiment thereof, the present invention provides a pharmaceutical composition comprising an anticancer agent as an active ingredient, in addition to phenformin or a pharmaceutically acceptable salt thereof, and 2-deoxy-D-glucose.

In this context, a pharmaceutical composition comprising an anticancer agent as an active ingredient in addition to phenformin hydrochloride and 2-deoxy-D-glucose may be in the form of a single dose unit containing the three active ingredients altogether, or in the form of three separate dose units containing the three active ingredients respectively. When the pharmaceutical composition is in the form of three dose units corresponding to the two active ingredients, they may be administered simultaneously or at time intervals so that they coexist in the body acting in synergy with one another. For example, exerting an enhanced therapeutic effect in terms of the alleviation or improvement of symptoms, the reduction of the scope of disease, the retardation or delay of disease progression, the improvement, alleviation or stabilization of disease state, partial or full recovery, the prolongation of survival, or other beneficial therapeutic results.

So long as it is known in the art, any anticancer agent may be used. For example, agents for use in chemotherapy, immunotherapy and gene therapy, including alkylating agents, metabolism inhibitors, natural agents, hormones, antagonists, and biological agents can be applied.

Exemplary among the anticancer agents useful in the present invention are nitrogen mustard, imatinib, oxaliplatin, ritoxmab, erlotinib, neratinib, lapatinib, gefitinib, vandetanib, nilotinib, semaxanib, bosutinib, axitinib, cediranib, lestaurtinib, trastuzumab, gefinitib, bortezomib, sunitinib, carboplatin, sorafenib, bevacizumab, cisplatin, cetuximab, viscumalbum, asparagenase, tretinoin, hydroxycarbamide, dasatinib, estramustine, gemtuxumab ozogamicin, Ibritumomab tiuxetan, heptaplatin, methylaminolevulinic acid, amsacrine, alemtuzumab, procarbazine, alprostadil, holmium nitrate chitosan, gemcitabine, doxifluridine, pemetrexed, tegafur, capecitabine, gimeracil, oteracil, azacytidine, methotrexate, uracil, cytarabine, fluorouracil, fludarabine, enocitabine, flutamide, decitabine, mercaptopurine, thioguanine, cladribine, carmofur, raltitrexed, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, pepromycin, temsirolimus, temozolomide, busulfan, ifosfamide, cyclophosphamide, melphalan, altretamine, dacabazine, thiotepa, nimustine, chlorambucil, mitolactol, leucovorin, tretinoin, exemestane, aminoglutethimide, anagleride, navelbine, fadrazole, tamoxifen, toremifene, testolactone, anastrozole, letrozole, vorozole, bicalutamide, lomustine and carmustine.

Also, contemplated in accordance with a further aspect of the present invention is a method for treating cancer comprising administering to a subject an effective amount of phenformin or a pharmaceutically acceptable salt thereof and a glycolysis inhibitor.

In a preferred embodiment, the method for treating cancer is performed in such a manner that 2-deoxy-D-glucose is administered in advance of phenformin or a pharmaceutically acceptable salt thereof.

In another preferred embodiment, the 2-deoxy-D-glucose is in an immediate release form while phenformin or a pharmaceutically acceptable salt thereof is a delayed release form.

Phenformin salts, weight ratios of the active ingredients, and conditions for time-lag administration, which are described in the preferred embodiments of the pharmaceutical composition, are true of the method for treating cancer in accordance with the present invention, too.

In the present invention, phenformin or a pharmaceutically acceptable salt thereof acts in synergy with 2-deoxy-D-glucose, thus exhibiting more potent inhibitory activity against the growth of cancer cells, compared to individual ingredients. Further, the time-lag release composition of the present invention decreases blood lactic acid levels to significantly mitigate the adverse effect of lactic acidosis, as well as exerting high anticancer effects. In addition, the synergistic anticancer activity allows the individual drugs to be used in lower amounts, which leads to a reduction in the occurrence of adverse effects. Therefore, the pharmaceutical composition of the present invention guarantees a higher anticancer effect although using a lower amount of each of the ingredients, thus enjoying the advantage of reducing the adverse effects of drugs and exerting high therapeutic effects. In addition, the pharmaceutical composition of the present invention can be formulated to dosage forms effective for therapy, increasing the drug compliance of the subject.

Consequently, the pharmaceutical composition of the present invention can be very effectively applied to the therapy of various cancer diseases.

### DESCRIPTION OF DRAWINGS

The above and other objects, features and advantages of the present invention will be more clearly understood from the following detailed description taken in conjunction with the accompanying drawings, in which:

FIG. 1 is a graph in which the degrees of activation of AMPK are depicted as assayed in Experimental Example 1. In the figure, 2-DG stands for 2-deoxy-D-glucose;

FIG. 2 is a graph showing inhibitory activity against tumor growth as measured in Experimental Example 4. In the figure, 2-DG stands for 2-deoxy-D-glucose; and

FIG. 3 is a graph showing blood lactic acid levels as measured in Experimental Example 5. In the figure, 2-DG stands for 2-deoxy-D-glucose.

### MODE FOR INVENTION

A better understanding of the present invention may be obtained through the following examples which are set forth to illustrate, but are not to be construed as the limit of the present invention.

### Experimental Example 1

### Assay of Combination Composition Comprising Phenformin Hydrochloride and 2-Deoxy-D-glucose for AMPK Activation

AMPK activation occurs when the generation of ATP, an energy source necessary for the survival of cancer cells, is inhibited. An examination was made of a synergistic effect of phenformin and 2-deoxy-D-glucose on AMPK activation, compared to individuals.

In this regard, AMPKα (5′-AMP-activated protein kinase alpha) activation was measured in the MCF7 human breast cancer cells using the AMPKα immunoassay kit (Invitrogen, catalog No. KHO0651).

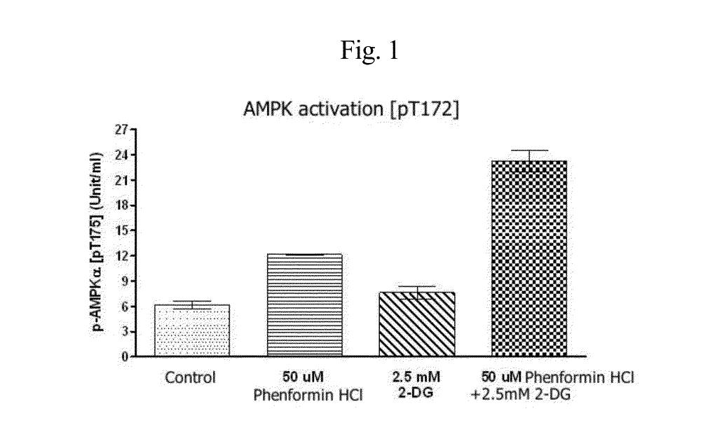

Briefly, MCF7 cells (purchased from the Korean Cell Line Bank) were grown in DMEM (Dulbeco's Modified Eagle Medium) (purchased from Gibco Life Technologies USA) supplemented with 10% (v/v) bovine calf serum, and seeded at a density of 5×10⁵ cells/well into 6-well plates before incubation in a 5% CO₂ incubator (culture temperature: 37° C., pH: 7.0˜7.4). The cell cultures were treated for 24 h with 2.5 mM 2-deoxy-D-glucose or 50 μM phenformin HCl, or a combination of 2-deoxy-D-glucose and phenformin HCl at the same concentrations. Then, the cells were lysed using the AMPKα immunoassay kit (Invitrogen, catalog No. KHO0651) according to the manufacturer's instruction. From 20 μg of the cell lysate obtained by a protein assay, the phosphorylation of AMPKα at the threonin 172 residue (Thr172) was quantified, and the results are given in Table 1 and FIG. 1.

TABLE 1
Assay for AMPK Activation

| | | | | 2-Deoxy- |
|---|---|---|---|---|
| | | 2-Deoxy-D- | Phenformin | D-glucose + |
| | Control | glucose | HCl | Phenformin HCl |
| Conc. | 0 | 2.5 mM | 50 nM | 2.5 mL |
| | | | | 2-deoxy-D- |
| | | | | glucose + |
| | | | | 50 μM |
| | | | | phenformin HCl |
| P-AMPK | 6.16 ± 1.0 | 7.62 ± 1.2 | 12.14 ± 2.0 | 23.21 ± 3.8 |
| (Unit/ml) | | | | |
| Normalized | 0 | 1.46 | 5.98 | 17.05 |
| to Control | | | | |
| (P-AMPK) | | | | |

As is understood from the data of Table 1 and FIG. 1, AMPK activation was surprisingly increased by a combination of 2-deoxy-D-glucose and phenformin HCl about 11.6-fold higher, compared to 2-deoxy-D-glucose alone, and about 3-fold higher, compared to phenformin HCl alone, as analyzed for the phosphorylation of AMPKα at the threonine 172 residue (Thr172).

Thus, a combination of 2-deoxy-D-glucose and phenformin produced a significant increase in AMPK activity, compared to their individual use. That is, acting in synergy with each other, 2-deoxy-D-glucose and phenformin were observed to exhibit improved anticancer activity associated with AMPK activation.

### Experimental Example 2

### Inhibitory Activity of Combination Composition of Phenformin HCl and 2-Deoxy-D-Glucose Against Cancer Cells

A combination composition of phenformin HCl and 2-deoxy-D-glucose was assayed for inhibitory activity against cancer cells including the human breast cancer cell line MCF7 and the human stomach cancer cell line NCI-N87, as follows.

The inhibitory activity of the combination composition of phenformin HCl and 2-deoxy-D-glucose against cancer cells was evaluated in the human breast cancer cell line MCF7 and human stomach cancer cell line NCI-N87 (both purchased from the Korean Cell Line Bank) by measuring cell viability (%) with the MTT reagent (3-(4,5-dimethylthiazole-2-yl)-2,5-ditetrazoliumbromide).

Briefly, MCF7 and NCI-N87 cells were grown at a density of 5,000 cells/well in DMEM and RPMI1640 media (both purchased from Gibco Life Technologies, USA), respectively, both supplemented with 10% (v/v) bovine calf serum (BCS), on 96-well plates for 16 h (Temp.: 37° C., pH: 7.0˜7.4). Then, the MCF7 cells were incubated for 48 h with 2.5 mM 2-deoxy-D-glucose and 100 μM phenformin HCl, solely or in combination while NCI-N87 cells were incubated for 48 h with 2.5 mM 2-deoxy-D-glucose and 700 μM phenformin HCl, solely or in combination (Temp.: 37° C., pH: 7.0˜7.4). To quantitate viable cells after incubation with 2-deoxy-D-glucose and phenformin HCl, the cell cultures were further incubated for 3 h in the presence of MTT. The formazan crystals thus formed were dissolved with DMSO (dimethyl sulfoxide) before reading absorbance at 560 nm.

After the 48 h incubation, viable cells were counted on the well plates treated with 2-deoxy-D-glucose and phenformin, and expressed as % cell growth inhibition relative to the count of the viable cells on the well plates treated with neither 2-deoxy-D-glucose nor phenformin. Results obtained by treating the cells with 2-deoxy-D-glucose alone, phenformin HCl alone, a combination of phenformin HCl and 2-deoxy-D-glucose are summarized in Table 2, below.

TABLE 2
% Cell Growth Inhibition in MCF7 and NCI-N87 Cells

| | | | | 2-Deoxy-D- |
|---|---|---|---|---|
| Cell | | 2-Deoxy-D- | Phenformin | glucose + |
| Line | Test Reagent | glucose | HCl | Phenformin HCl |
| MCF7 | Conc. | 2.5 mM | 100 μM | 2.5 mM + 100 μM |
| | Cell Growth | 44.2 | 14.3 | 83.9 |
| | Inhibition | | | |
| | (%) | | | |
| NCI- | Conc. | 2.5 mM | 700 μM | 2.5 mM + 700 μM |
| N87 | Cell Growth | 15.1 | 52.5 | 98.1 |
| | Inhibition | | | |
| | (%) | | | |

As is apparent from data of Table 2, the growth of both the cancer cell lines MCF7 and NCI-N87 was inhibited to higher extent when they were treated with a combination of phenformin and 2-deoxy-D-glucose than with individuals thereof. The combination of phenformin and 2-deoxy-D-glucose inhibited the growth MCF cells by about 2 folds than 2-deoxy-D-glucose alone, and by about 6 folds than phenformin HCl alone. With regard to NCI-N87 cells, the inhibitory activity of the combination was about 6.5-fold and about 2-fold higher than 2-deoxy-D-glucose alone and phenformin HCl alone, respectively. The results suggest that a combination of phenformin and 2-deoxy-D-glucose exerts a significant therapeutically synergistic effect on cancer cells.

Meanwhile, the inhibitory effect of 2-deoxy-D-glucose on the breast cancer cells was found to be about 3 times as large as that on the stomach cancer cells. To compensate for the relatively low effect on stomach cancer cells, phenformin HCl was used at higher concentrations for the stomach cancer cells. At a 7-fold increased concentration, phenformin HCl exhibited an approximately 4-fold higher inhibitory effect on the stomach cancer cells than the breast cancer cells.

Like this, phenformin HCl alone was observed to elicit an inhibitory effect which was relatively small in comparison to the amount used. When used alone, either 2-deoxy-D-glucose or phenformin HCl does not have noticeable inhibitory activity against the growth of stomach cancer cells, compared to breast cancer cells.

In contrast, when used in combination in such a manner that phenformin HCl varied in concentration, with a constant concentration given to 2-deoxy-D-glucose, as in their individual use, the two drugs were found to exert high inhibitory effects on stomach cancer cells as well as breast cancer cells. Thus, the use of the two drugs in combination was significantly effective for the therapy of the cancer even though it was resistant to their individual use. In addition, higher therapeutic effects are possible by adjusting the composition ratio and content of each of the drugs.

### Experimental Example 3

### Inhibitory Effect of Phenformin HCl and 2-Deoxy-D-Glucose on Cancer Cell Growth by Composition Ratio Thereof

When the human breast cancer cell line MCF7 and the human stomach cancer cell line NCI-N87 were treated with various concentrations of phenformin and 2-deoxy-D-glucose, % inhibition of cancer cell growth was measured. The same procedure as in Experimental Example 2 was repeated, with the exception that 2-deoxy-D-glucose and phenformin HCl were used at different concentrations. The results are summarized in Tables 3 and 4, below.

TABLE 3
Inhibitory Effect on Growth of MCF7 Cells

| | | Concentration of | | | |
|---|---|---|---|---|---|
| Tumor Growth | | 2-Deoxy-D-glucose | | | |
| Inhibition (%) | | 0 | 100 μM | 500 μM | 2 mM |
| Concentration of | 0 | 0 | 0 | 5 | 31 |
| Phenformin | 10 μM | 9 | 11 | 29 | 55 |
| | 50 μM | 12 | 20 | 41 | 62 |
| | 100 μM | 14 | 22 | 48 | 66 |
| | 200 μM | 18 | 30 | 52 | 69 |
| | 400 μM | 31 | 41 | 60 | 73 |
| | 1 mM | 58 | 62 | 70 | 77 |

TABLE 4
Inhibitory Effect on Growth of NCI-N87 Cells

| | | Concentration of | | | |
|---|---|---|---|---|---|
| Tumor Growth | | 2-Deoxy-D-glucose | | | |
| Inhibition (%) | | 0 | 100 μM | 500 μM | 2 mM |
| Concentration of | 0 | 0 | 3 | 6 | 32 |
| Phenformin | 10 μM | 22 | 29 | 52 | 69 |
| | 50 μM | 24 | 31 | 51 | 70 |
| | 100 μM | 24 | 32 | 56 | 70 |
| | 200 μM | 24 | 33 | 57 | 72 |
| | 400 μM | 26 | 35 | 59 | 75 |
| | 1 mM | 39 | 47 | 76 | 91 |

As is understood from the data of Tables 3 and 4, both phenformin HCl and 2-deoxy-D-glucose produced dose-dependent increases in inhibitory activity against each cancer cell line. In comparison to the sum (30%) of individual uses of 100 μM phenformin HCl (24%) and 500 μM 2-deoxy-D-glucose (6%), the use of the drugs in combination inhibited cell growth by 56%, which is about 2-fold higher. Like this, the drugs were found to act in synergy with each other, exerting significantly increased inhibitory activity against cancer cells, compared to the sum of their individual uses.

As for phenformin HCl, its inhibitory effects were higher even at relatively low concentrations, compared to 2-deoxy-D-glucose. In full consideration of the difference in inhibitory activity therebetween, the synergistic effect of phenformin HCl and 2-deoxy-D-glucose was detected in a broad range of a weight ratio of 1:200 to 10:1.

### Comparative Experimental Example 1

### Inhibitory Effect of Metformin and 2-Deoxy-D-Glucose on Cancer Cell Growth by Composition Ratio Thereof

In order to confirm the synergistic effect of the present invention, metformin hydrochloride, a biguanide drug which has the most similar in structure and effect to phetformin, was used instead, in combination with 2-deoxy-D-glucose in assaying cancer cell growth inhibition (%). The same procedure as in Experimental Example 3 was repeated in the breast cancer cell line MCF7 and the colorectal cancer cell line HCT116 (both purchased from the Korean Cell Line Bank), with the exception that the materials and concentrations indicated in Tables 5 and 6 were employed.

TABLE 5
Inhibitory Effect on Growth of MCF7 Cells

| | | Concentration | | | |
|---|---|---|---|---|---|
| Tumor Growth | | 2-Deoxy-D-Glucose | | | |
| Inhibition (%) | | 0 | 2.5 mM | 5.0 mM | 20.0 mM |
| Metformin HCl | 0 | 0 | 58 | 76 | 91 |
| Concentration | 0.625 mM | 9 | 76 | 88 | 96 |
| | 1.25 mM | 22 | 80 | 90 | 98 |
| | 2.5 mM | 24 | 82 | 90 | 98 |
| | 5.0 mM | 30 | 85 | 90 | 97 |
| | 10.0 mM | 38 | 88 | 92 | 96 |
| | 20.0 mM | 61 | 90 | 91 | 99 |

TABLE 6
Inhibitory Effect on Growth of HCT116 Cells

| | | Concentration | | | |
|---|---|---|---|---|---|
| Tumor Growth | | 2-Deoxy-D-glucose | | | |
| Inhibition (%) | | 0 | 2.5 mM | 5.0 mM | 20.0 mM |
| Metformin HCl | 0 | 0 | 62 | 79 | 93 |
| Concentration | 0.625 mM | 7 | 63 | 79 | 94 |
| | 1.25 mM | 11 | 66 | 80 | 95 |
| | 2.5 mM | 40 | 78 | 90 | 97 |
| | 5.0 mM | 64 | 92 | 95 | 99 |
| | 10.0 mM | 63 | 96 | 97 | 100 |
| | 20.0 mM | 66 | 97 | 98 | 100 |

When used in combination with 2-deoxy-D-glucose, as shown in Tables 5 and 6, metformin HCl, similar to phenformin HCl, although at high concentrations, did not elicit synergistic effects at all, as opposed to the data of Tables 3 and 4 of Experimental Example 3. Rather, the combination of metformin HCl and 2-deoxy-D-glucose was lower in inhibitory activity against the cancer cells than the sum of their individual uses.

In spite of a biguanide drug having a structure the most similar to that of phenformin HCl, metformin HCl was found to not act in synergy with 2-deoxy-D-glucose. Therefore, a synergistic effect with 2-deoxy-D-glucose is not common to biguanide drugs, but is peculiar to phenformin HCl.

### Experimental Example 4

### Inhibitory Activity Against Tumor Growth by Simultaneous or Time-Lag Administration of Phenformin HCl and 2-Deoxy-D-Glucose

The human colorectal cancer cell line HCT116 (purchased from the Korean Cell Line Bank) was subcutaneously injected at a concentration of 4×10⁶ cells/0.1 mL into the right flank of Balb/c athymic nude mice. When the tumor grew to a volume of 140 mm³, the mice were divided into groups of five so that sizes of the tumor were distributed uniformly over the groups, followed by oral administration of test materials once a day for 23 days.

The test materials were 40 mM citrate buffer (pH 6.0) containing 5% (w/v) Arabic gum for group 1 (excipient control), a combination of phenformin HCl 50 mg/kg [symbol] 2-deoxy-D-glucose 750 mg/kg for group 2 (administered simultaneously), a combination of 2-deoxy-D-glucose 750 mg/kg and phenformin HCl 50 mg/kg for group 3 (administered with a time lag), a combination of phenformin HCl 50 mg/kg and 2-deoxy-D-glucose 1,000 mg/kg for group 4 (simultaneously administrated), and a combination of 2-deoxy-D-glucose 1,000 mg/kg and phenformin HCl 50 mg/kg for group 5 (administered with a time lag). In the time-lag administered groups 3 and 5,2-deoxy-D-glucose was administered 2 h in advance of phenformin HCl.

Tumor sizes were measured once every two or three days using a caliper, and determined according to the following math formula. However, when the tumor size reached 3000 mm³, the mice was killed even before completion of the experiment.

Tumor volume=(Long Axis×Short Axis×height)/2 <Math Formula>

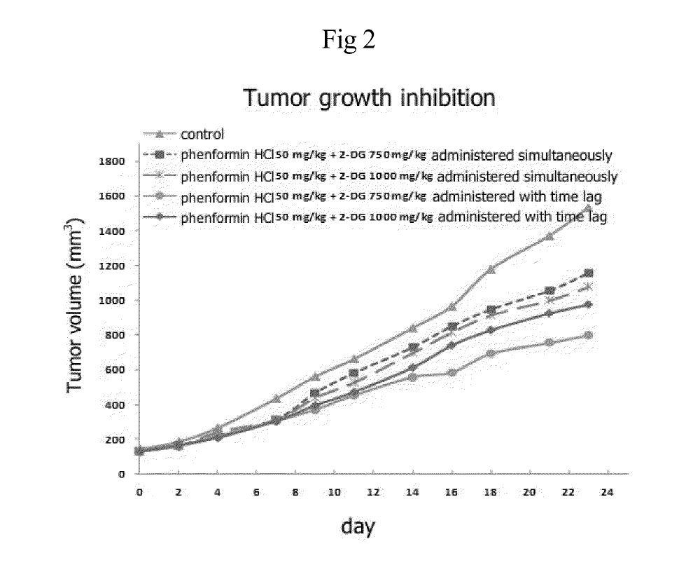

Tumor volumes measured by day, and tumor growth inhibition (TGI) (%) for each group, relative to the excipient control, are given in Table 7, and FIG. 2.

TABLE 7
Inhibitory Effect on Tumor Growth by Simultaneous and Time-Lag
Administration of Phenformin and 2-Deoxy-D-Glucose
(day 23, after completion of administration)

| | | Administration | Tumor Vol. | TGI |
|---|---|---|---|---|
| Group | Content | Amount | (mm³) | (%) |
| 1 | Excipient Control | 0 | 1534.1 ± 547.2 | 0.0 |
| 2 | 2-deoxy-D-glucose + | 2-deoxy-D- | 1157.6 ± 313.2 | 24.5 |
| | phenformin HCl | glucose | | |
| | administered | 750 mg/kg + | | |
| | simultaneously | | | |
| 3 | 2-deoxy-D-glucose + | phenformin | 798.8 ± 217.7 | 47.9 |
| | phenformin HCl | HCl 50 mg/kg | | |
| | administered with | | | |
| | time lag | | | |
| 4 | 2-deoxy-D-glucose + | 2-deoxy-D- | 1076.4 ± 328.7 | 29.8 |
| | phenformin HCl | glucose | | |
| | administered | 1,000 mg/kg + | | |
| | simultaneously | | | |
| 5 | 2-deoxy-D-glucose + | phenformin | 974.9 ± 208.9 | 36.5 |
| | phenformin HCl | HCl 50 mg/kg | | |
| | administered with | | | |
| | time lag | | | |

As shown in Table 7 and FIG. 2, tumor volumes were measured to be 1534.1±547.2 mm³ in Group 1, 1157.6±313.2 mm³ in Group 2, 798.8±217.7 mm³ in Group 3, 1076.4±328.7 mm³ in Group 4, and 974.9±208.9 mm³ in Group 5. In terms of TGI (%), inhibitory effects were detected in all the groups administered with both phenformin and 2-deoxy-D-glucose. Groups 3 and 5 in which 2-deoxy-D-glucose was administered 2 h before phenformin exhibit further increased tumor growth inhibition, compared to Groups 2 and 4 in which the two drugs were administered simultaneously. Particularly, a higher inhibitory effect was observed in Group 3 than Group 4 which was administered simultaneously with a higher dose of 2-deoxy-D-glucose, and phenformin, indicating that time-lag administration brings about a higher inhibitory effect even at a lower dose.

As described above, an increase in anticancer activity by time-lag administration of a combination of the anticancer drugs was a surprising effect first demonstrated in the present invention. Hence, the two active ingredients act in synergy with each other, exerting a further increased therapeutic effect upon administration with a time lag (or release with a time lag after simultaneous administration).

### Experimental Example 5

### Blood Lactate Level after Simultaneous and Time-Lag Administration of Phenformin HCl and 2-Deoxy-D-Glucose

The same procedure as in Experimental Example 4 was repeated, with the exception that the following doses were employed.

As an excipient control, 40 mM citrate buffer (pH 6.0) containing 5% (W/V) Arabic gum was used. While the dose of phenformin HCl was maintained constantly at 50 mg/kg, 2-deoxy-D-glucose was administered in an amount of 750 mg/kg, 1,000 mg/kg, or 1,500 mg/kg for 23 days. Blood lactate levels were measured after they were administered simultaneously or with a time lag.

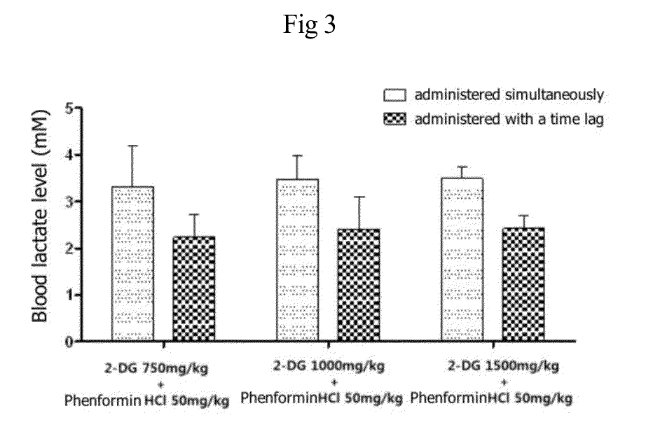

Measurements of blood lactate levels (mM) are depicted in FIG. 3.

As can be seen in FIG. 3, blood lactate was measured at low levels after both simultaneous and time-lag administration of phenformin HCl and 2-deoxy-D-glucose, with a further decrease by time-lag administration.

Therefore, co-administration of phenformin HCl and 2-deoxy-D-glucose with a time lag produces a synergistic anticancer effect, with a significant decrease in lactic acidosis, a problem with phenformin.

As described above, the pharmaceutical composition comprising phenformin or a pharamceutically acceptable salt thereof, and 2-deoxy-D-glucose in accordance with the present invention shows high inhibitory effects on the growth of cancer cells, with a further increase in the inhibitory activity against cell growth upon the time-lag administration thereof. Particularly, it is found in the present invention that the problem with phenformin, that is, an increased blood lactate level by phenformin, can be surprisingly solved when they are administered with a time lag. Accordingly, the pharmaceutical composition of the present invention can be effectively used as an anticancer or antitumor agent for the treatment of various cancers, without producing adverse effects.

### Example 1

### Preparation of Tablet Containing Phenformin HCl and 2-Deoxy-D-glucose

After 50.0 g of phenformin HCl, 300 g of 2-deoxy-D-glucose and 200.0 g of microcrystalline cellulose were sieved with respective sieve No. 20 meshes, they were mixed for 20 min in V-mixer (Cheil Company, Korea). Separately, 25 g of hydroxypropyl cellulose and 10 g of colloidal silicon dioxide were sieved through sieve No. 35 meshes, and added to and mixed for 10 min with the mixture. Finally, 5 g of stearic acid was sieved through a sieve No. 35 mesh, and added to and mixed for 3 min with the mixture. Subsequently, the final mixture was pressed into tablets containing phenformin HCl and 2-deoxy-D-glucose. Then, the tablets were coated with a film made of 15 g of Opadry OY-C-7000A using High Coater (SFC-30N Sejong Machinery, Korea) to afford final tables in which 50 mg of phenformin HCl and 300 mg of 2-deoxy-D-glucose were contained per tablet.

### Example 2

### Preparation of Tablet Containing Phenformin HCl and 2-Deoxy-D-Glucose

After 100.0 g of phenformin HCl, 50 g of 2-deoxy-D-glucose, and 200.0 g of microcrystalline cellulose were sieved with respective sieve No. 20 meshes, they were mixed for 3 min in a high-speed mixer (YC-SMG-10J, Yenchen, Taiwan). Separately, 25 g of povidone was dissolved in 150 g of isopropanol to give a binder which was then blended with the mixture for 3 min in the high-speed mixer. The resulting blend was dried in a steam drier, followed by sieving through sieve No. 20 to afford granules.

Separately, 10 g of colloidal silicon dioxide was sieved through sieve No. 35, added to the mixture and mixed for 10 min. Finally, 5 g of stearic acid was sieved through sieve No. 35, added to the mixture and mixed for 3 min. The final blend was pressed into tablets containing phenformin HCl and 2-deoxy-D-glucose. Then, the tablets were coated with a film made of 20 g of Opadry OY-C-7000A using High Coater (SFC-30N Sejong Machinery, Korea) to afford final tables in which 100 mg of phenformin HCl and 50 mg of 2-deoxy-D-glucose were contained per tablet.

### Example 3

### Preparation of Sustained Release Tablet Containing Phenformin HCl and 2-Deoxy-D-glucose

After 50.0 g of phenformin HCl, 300 g of 2-deoxy-D-glucose, 50.0 g of dicalcium phosphate, and 350.0 g of polyethylene oxide (molecular weight 5 millions, Dow Chemical, USA) were each sieved through sieve No. 20, they were mixed for 45 min using V-mixer (Cheil Company, Korea). Separately, 30 g of hydroxypropyl cellulose and 10 g of colloidal silicon dioxide were sieved through sieve No. 35, added to the mixture, and mixed for 45 min. Finally, 5 g of magnesium stearate was sieved through sieve No. 35, and blended with the mixture for 3 min. The resulting blend was pressed into tablets containing phenformin HCl and 2-deoxy-D-glucose. Then, the tablets were coated with a film made of 20 g of Opadry OY-C-7000A using High Coater (SFC-30N Sejong Machinery, Korea) to afford final tables in which 50 mg of phenformin HCl and 300 mg of 2-deoxy-D-glucose were contained per tablet.

### Example 4

### Preparation of Capsule Containing Phenformin HCl and 2-Deoxy-D-glucose

After 100 g of phenformin HCl, 400 g of 2-deoxy-D-glucose and 272 g of microcrystalline cellulose were each sieved through sieve No. 20, they were mixed in V-mixer (Cheil Company, Korea) for 60 min. Separately, 8 g of colloidal silicon dioxide and 16 g of sodium starch glycolate were sieved through sieve No. 35, added to the mixture, and mixed for 60 min. Finally, 4 g of stearic acid was sieved through sieve No. 35, and blended with the mixture for 3 min.

Subsequently, the final blend was loaded to capsules so that 25 mg of phenformin HCl and 100 mg of 2-deoxy-D-glucose were contained per capsule.

### Example 5

### Preparation of Tablet Containing Phenformin HCl and 2-Deoxy-D-glucose

After 50 g of phenformin HCl, 600 g of 2-deoxy-D-glucose, 25 g of microcrystalline cellulose and 85 g of dicalcium phosphate were each sieved through sieve No. 20, they were mixed in a high-speed mixer (YC-SMG-10J, Yenchen, Taiwan) for 3 min. 20 g of povidone was dissolved in 120 g of isopropanol to give a binder which was then then blended with the mixture for 3 min in the high-speed mixer. The resulting blend was dried in a steam drier, followed by sieving through sieve No. 20 to afford granules.

Separately, 5 g of colloidal silicon dioxide, and 10 g of sodium starch glycolate were sieved through sieve No. 35, added to the mixture, and mixed for 10 min Finally, 5 g of magnesium stearate was sieved through sieve No. 35, and blended with the mixture for 3 min. The resulting blend was pressed into tablets containing phenformin HCl and 2-deoxy-D-glucose. Then, the tablets were coated with a film made of 20 g of Opadry OY-C-7000A using High Coater (SFC-30N Sejong Machinery, Korea) to afford final tables in which 50 mg of phenformin HCl and 600 mg of 2-deoxy-D-glucose were contained per tablet.

### Example 6

### Preparation of Tablet Containing Phenformin HCl and 2-Deoxy-D-glucose

After 150.0 g of phenformin HCl, 600 g of 2-deoxy-D-glucose, and 145 g of microcrystalline cellulose were each sieved through sieve No. 20, they were mixed in V-mixer (Cheil Company, Korea) for 20 min. Separately, 30 g of copovidone (Kollidon VA64, BASF, Germany), and 5 g of colloidal silicon dioxide were sieved through sieve No. 35, added to the mixture, and mixed for 10 min. Again, this mixture was mixed with 20 g of sodium starch glycolate for 5 min. Finally, 6 g of magnesium stearate was sieved through sieve No. 35, and blended with the mixture for 3 min. The resulting blend was pressed into tablets containing phenformin HCl and 2-deoxy-D-glucose. Then, the tablets were coated with a film made of 20 g of Opadry OY-C-7000A using High Coater (SFC-30N Sejong Machinery, Korea) to afford final tables in which 150 mg of phenformin HCl and 600 mg of 2-deoxy-D-glucose were contained per tablet.

### Example 7

### Preparation of Sustained-Release Tablet Containing Phenformin HCl and 2-Deoxy-D-glucose

After 50 g of phenformin HCl, 300 g of 2-deoxy-D-glucose, 10 g of microcrystalline cellulose and 300 g of hypromellose (Mw 100,000, Shin-Etsu, Japan) were each sieved through sieve No. 20, they were mixed in V-mixer (Cheil Company, Korea) for 45 min. 30 g of copovidone (Kollidon VA-64, BASF, Germany), and 5 g of colloidal silicon dioxide were sieved through sieve No. 35, added to the mixture, and mixed for 45 min. Finally, 5 g of stearic acid was sieved through sieve No. 35, and blended with the mixture for 3 min. The resulting blend was pressed into tablets containing phenformin HCl and 2-deoxy-D-glucose. Then, the tablets were coated with a film made of 15 g of Opadry OY-C-7000A using High Coater (SFC-30N Sejong Machinery, Korea) to afford final tables in which 50 mg of phenformin HCl and 300 mg of 2-deoxy-D-glucose were contained per tablet.

### Example 8

### Preparation of Time-Lag Release Agent Containing Phenformin HCl and 2-Deoxy-D-glucose: Formulation for Co-Package Kit

1) Preparation of Early-Release 2-deoxy-D-glucose Tablet

After 500.g g of 2-deoxy-D-glucose and 140.0 g of microcrystalline cellulose were sieved through respective sieve No. 20 meshes, they were mixed for 20 min in V-mixer (Cheil Company, Korea). Separately, 42.0 g of hydroxypropyl cellulose, and 10.0 g of colloidal silicon dioxide were sieved through sieve No. 35, added to the mixture, and mixed for 10 ml. Finally, 8.0 g of stearic acid was sieved through a sieve No. 35, added to the mixture and mixed for 3 min. The resulting blend was pressed into a tablet containing 500 mg of 2-deoxy-D-glucose.

2) Preparation of Late-Release Phenformin HCl Tablet

After 50.0 g of phenformin HCl ad 67.0 g of microcrystalline cellulose were each sieved through sieve No. 20, they were mixed in V-mixer (Cheil Company, Korea) for 20 min. Separately, 3.0 g of hydroxypropyl cellulose and 0.5 g of colloidal silicon dioxide were sieved through sieve No. 35, added to the mixture, and mixed for 10 min. Finally, 0.5 g of stearic acid was sieved through sieve No. 35, and blended with the mixture for 3 min. Subsequently, the resulting blend was compressed into tablets containing phenformin HCl. The tablets were coated with a coating solution of 9.0 g of hypromellose (15 cps), 3.0 g of hydroxypropylcellulose, 1.6 g of titanium dioxide, 1.0 g of polyethylene glycol 6,000, and 0.4 g of talc in a mixture of 50:50 ethanol-methylene chloride in High Coater (SFC-30N, Sejong Machinery, Korea) to afford coated tablets in which 50 mg of phenformin HCl was contained per tablet.

3) Package

One early-release 2-deoxy-D-glucose tablet prepared in 1) and one late-release phenformin HCl tablet prepared in 2) were co-packed in a blister packing machine.

### Example 9

### Preparation of Timed Release Formulation Containing Phenformin HCl and 2-Deoxy-D-glucose: Press-Coated Tablet

1) Preparation of Early-Release 2-deoxy-D-glucose Granule

After 500.0 g of 2-deoxy-D-glucose and 140.0 g of microcrystalline cellulose were each sieved through sieve No. 20, they were mixed in V-mixer (Cheil Company, Korea) for 20 min. Separately, 42.0 g of hydroxypropyl cellulose, and 10.0 g of colloidal silicon dioxide were sieved through sieve No. 35, added to the mixture, and mixed for 10 min. Finally, 8.0 g of stearic acid was sieved through sieve no. 35, added to the mixture, and blended for 3 min to produce early release granules containing 500 mg of 2-deoxy-D-glucose.

2) Preparation of Late-Release Phenformin HCl Tablet

After 50.0 g of phenformin HCl and 67.0 g of microcrystalline cellulose were each sieved through sieve No. 20, they were mixed in V-mixer (Cheil Company, Korea) for 20 min. Separately, 3.0 g of hydroxypropyl cellulose was dissolved in 30.0 g of 70% ethanol to give a binder solution which was then blended with the mixture in a high-speed mixer (YC-SMG-10J, Yenchen, Taiwan), and dried. The granules thus formed were sieved through sieve No. 20, and mixed for 10 min with 4.0 g of crospovidone and 0.5 g of colloidal silicon dioxide. Finally, 0.5 g of magnesium stearate was sieved through sieve No. 35, and blended with the mixture for 3 min. Subsequently, the resulting blend was compressed into bare tablets containing phenformin HCl. Then, the tablets were coated with a film made of 15.0 g of Opadry OY-C-7000A using High Coater (SFC-30N Sejong Machinery, Korea) to afford cored tables in which 50 mg of phenformin HCl was contained per tablet

3) Preparation of Press-Coated Tablet

Using a press-coated tableting machine (RUD-1, Kilian, Germany), the early-release 2-deoxy-D-glucose granules prepared in 1) (700.0 mg per tablet) and the late-release phenformin tablet prepared in 2) (140.0 mg per tablet) were formulated into a press-coated tablet configured to release 2-deoxy-D-glucose first, and then phenformin.

### Example 10

### Preparation of Timed Release Formulation Containing Phenformin HCl and 2-Deoxy-D-glucose: Granule+Tablet

1) Preparation of Early-Release 2-deoxy-D-glucose Granules

After 500.0 g of 2-deoxy-D-glucose, and 182.0 g of microcrystalline cellulose were each sieved through sieve No. 20, they were mixed in V-mixer (Cheil Company, Korea) for 20 min. Separately, 10.0 g of colloidal silica was sieved through sieve No. 35, added to the mixture, and mixed for 10 min. Finally, 8.0 g of stearic acid was sieved through sieve no. 35, added to the mixture, and blended for 3 min to produce early release granules containing 500 mg of 2-deoxy-D-glucose.

2) Preparation of Late-Release Phenformin HCl Tablet

After 50.0 g of phenformin HCl, and 67.0 g of microcrystalline cellulose were each sieved through sieve No. 20, they were mixed in V-mixer (Cheil Company, Korea) for 20 min. Separately, 3.0 g of hydroxypropyl cellulose was dissolved in 30.0 g of 70% ethanol to give a binder solution which was then blended with the mixture in a high-speed mixer (YC-SMG-10J, Yenchen, Taiwan), and dried. The granules thus formed were sieved through sieve No. 20, and mixed for 10 min with 4.0 g of crospovidone and 0.5 g of colloidal silicon dioxide. Finally, 0.5 g of magnesium stearate was sieved through sieve No. 35, and blended with the mixture for 3 min. Subsequently, the resulting blend was compressed into bare tablets containing phenformin HCl. The tablets were coated with a coating solution of 9.0 g of hypromellose (15 cps), 3.0 g of hydroxypropylcellulose, 1.6 g of titanium dioxide, 1.0 g of polyethylene glycol 6,000, and 0.4 g of talc in a mixture of 50:50 ethanol-methylene chloride in High Coater (SFC-30N, Sejong Machinery, Korea) to afford coated tablets in which 50 mg of phenformin HCl was contained per tablet.

3) Package

The early-release 2-deoxy-D-glucose granules prepared in 1) and one late-release phenformin HCl tablet prepared in 2) were loaded together in a pouch sac.

### Example 11

### Preparation of Timed-Release Formulation Containing Phenformin HCl and 2-Deoxy-D-glucose: Press-Coated Tablet

The formulation was prepared in the same manner as in Example 9, with the exception that the late-release phenformin HCl tablets were made as follows.

After 50.0 g of phenformin HCl and 52.0 g of microcrystalline cellulose were each sieved through sieve No. 20, they were mixed in V-mixer (Cheil Company, Korea) for 20 min. Separately, 3.0 g of hydroxypropyl cellulose was dissolved in 30.0 g of 70% ethanol to give a binder solution which was then blended with the mixture in a high-speed mixer (YC-SMG-10J, Yenchen, Taiwan), and dried. The granules thus formed were sieved through sieve No. 20, and mixed for 10 min with 19.0 g of hypromellose 2208 (100,000 cp), and 0.5 g of colloidal silica. Finally, 0.5 g of magnesium stearate was sieved through sieve No. 35, and blended with the mixture for 3 min. Subsequently, the resulting blend was compressed into bare tablets containing phenformin HCl. The tablets were coated with a coating solution of 9.0 g of hypromellose (15 cps), 3.0 g of hydroxypropyl cellulose, 1.6 g of titanium dioxide, 1.0 g of polyethylene glycol 6,000, and 0.4 g of talc in a mixture of 50:50 ethanol-methylene chloride in High Coater (SFC-30N, Sejong Machinery, Korea) to afford coated tablets in which 50 mg of phenformin HCl was contained per tablet.

### Example 12

### Preparation of Timed-Release Formulation Containing Phenformin HCl and 2-Deoxy-D-glucose: Capsule

1) Preparation of Late-Release Phenformin HCl Pellet

In 200.0 g of ethanol were dissolved 50.0 g of phenformin HCl and 10.0 g of hypromellose 2910, and this solution was sprayed over 50.0 g of sugar spheres in a fluidized bed granulation coater (SFC-mini, Freund, Japan) to give phenformin HCl pellets. They were further coated with 15.0 g of Opadry OY-C-7000A afford pellets containing 50 mg of phenformin HCl (within 125 mg of pellets).

2) Preparation of Capsule

Together with 500.0 mg of 2-deoxy-D-glucose, 125.0 mg of the late-release phenformin HCl pellets prepared in 1) were loaded to a capsule with a size of 00.

### Example 13

### Preparation of Timed-Release Formulation Containing Phenformin HCl, 2-Deoxy-D-glucose, and Imatinib: Press-Coated Tablet

1) Preparation of Early-Release Granules Containing 2-deoxy-D-glucose, and Imatinib Mesylate

After 500.0 g of 2-deoxy-D-glucose, 100.0 g of imatinib mesylate, 60.0 g of lactose, and 30.0 g of microcrystalline cellulose were each sieved through sieve No. 20, they were mixed in V-mixer (Cheil Company, Korea) for 20 min. Separately, 20.0 g of hydroxypropyl cellulose, and 10.0 g of colloidal silicon dioxide were sieved through sieve No. 35, added to the mixture, and mixed for 10 min. Finally, 10.0 g of magnesium stearate was sieved through sieve No. 35, added to the mixture and blended for 3 min to afford early-release granules containing 500 mg of 2-deoxy-D-glucose and 100 mg of imatinib mesylate.

2) Preparation of Late-Release Phenformin HCl Tablet

After 50.0 g of phenformin HCl and 67.0 g of microcrystalline cellulose were each sieved through sieve No. 20, they were mixed in V-mixer (Cheil Company, Korea) for 20 min. Separately, 3.0 g of hydroxypropyl cellulose was dissolved in 30.0 g of 70% ethanol to give a binder solution which was then blended with the mixture in a high-speed mixer (YC-SMG-10J, Yenchen, Taiwan), and dried. The granules thus formed were sieved through sieve No. 20, and mixed for 10 min with 4.0 g of crospovidone and 0.5 g of colloidal silicon dioxide. Finally, 0.5 g of magnesium stearate was sieved through sieve No. 35, and blended with the mixture for 3 min. Subsequently, the resulting blend was compressed into bare tablets containing phenformin HCl. The tablets were coated with a coating solution of 9.0 g of hypromellose (15 cps), 3.0 g of hydroxypropyl cellulose, 1.6 g of titanium dioxide, 1.0 g of polyethylene glycol 6,000, and 0.4 g of talc in a mixture of 50:50 ethanol-methylene chloride in High Coater (SFC-30N, Sejong Machinery, Korea) to afford coated tablets in which 50 mg of phenformin HCl was contained per tablet.

3) Preparation of Press-Coated Tablet

Using a core tableting machine (RUD-1, Kilian, Germany), the early-release granules containing 2-deoxy-D-glucose granules and imatinib mesylate, prepared in 1) (730 mg per tablet) and the late-release phenformin tablet prepared in 2) (140.0 mg per tablet) were formulated into a press-coated tablet configured to release 2-deoxy-D-glucose and imatinib first, and then phenformin.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method for treating cancer comprising administering to a subject an effective amount of phenformin or a pharmaceutically acceptable salt thereof and a glycolysis inhibitor.

2. The method of claim 1, wherein the pharmaceutically acceptable salt of phenformin is phenformin HCl.

3. The method of claim 1, wherein the glycolysis inhibitor is 2-deoxy-D-glucose.

4. The method of claim 3, wherein phenformin or a pharmaceutically acceptable salt thereof and 2-deoxy-D-glucose are administered at a weight ratio of from 1:400 to 100:1.

5. The method of claim 4, wherein phenformin or a pharmaceutically acceptable salt thereof and 2-deoxy-D-glucose are administered at a weight ratio of from 1:200 to 10:1.

6. The method of claim 3, wherein phenformin or a pharmaceutically acceptable salt thereof and 2-deoxy-D-glucose are in an oral or non-oral dosage form.

7. The method of claim 1, further administering an anticancer agent.

8. The method of claim 7, wherein the anticancer agent is selected from the group consisting of nitrogen mustard, imatinib, oxaliplatin, ritoxmab, erlotinib, neratinib, lapatinib, gefitinib, vandetanib, nilotinib, semaxanib, bosutinib, axitinib, cediranib, lestaurtinib, trastuzumab, gefinitib, bortezomib, sunitinib, carboplatin, sorafenib, bevacizumab, cisplatin, cetuximab, viscumalbum, asparagenase, tretinoin, hydroxycarbamide, dasatinib, estramustine, gemtuxumab ozogamicin, Ibritumomab tiuxetan, heptaplatin, methylaminolevulinic acid, amsacrine, alemtuzumab, procarbazine, alprostadil, holmium nitrate chitosan, gemcitabine, doxifluridine, pemetrexed, tegafur, capecitabine, gimeracil, oteracil, azacytidine, methotrexate, uracil, cytarabine, fluorouracil, fludarabine, enocitabine, flutamide, decitabine, mercaptopurine, thioguanine, cladribine, carmofur, raltitrexed, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, pepromycin, temsirolimus, temozolomide, busulfan, ifosfamide, cyclophosphamide, melphalan, altretamine, dacabazine, thiotepa, nimustine, chlorambucil, mitolactol, leucovorin, tretinoin, exemestane, aminoglutethimide, anagleride, navelbine, fadrazole, tamoxifen, toremifene, testolactone, anastrozole, letrozole, vorozole, bicalutamide, lomustine and carmustine.

9. The method of claim 1, wherein the cancer is selected from the group consisting of uterine cancer, breast cancer, stomach cancer, brain cancer, rectal cancer, colon cancer, lung cancer, skin cancer, blood cancer, and liver cancer.

10. The method of claim 9, wherein the cancer is breast cancer, stomach cancer, or colon cancer.

11. The method of claim 3, wherein an effective amount of 2-deoxy-D-glucose is administered to a subject in advance of an effective amount of phenformin or a pharmaceutically acceptable salt thereof.

12. The method of claim 11, wherein phenformin or a pharmaceutically acceptable salt thereof is administered 0.25˜4.0 hours after 2-deoxy-D-glucose is administered.

13. The method of claim 3, wherein 2-deoxy-D-glucose is administered in an immediate release dosage form while phenformin or a pharmaceutically acceptable salt thereof is administered a delayed-release dosage form.

14. The method of claim 13, wherein the delayed-release dosage form is a sustained-release or a pulsed-release dosage form.

15. The method of claim 1, wherein the glycolysis inhibitor is a tyrosine kinase inhibitor.
